(19) United States
(12) Patent Application Publication
(10) Pub. No.: US 2009/0326299 A1
(43) Pub. Date: Dec. 31, 2009

(54) Use of a Catalyst Based on Zeolites in the Conversion of Oxygenates to Lower Olefins, and Associated Method

(75) Inventors: Arno Tissler, Tegernheim (DE); Rainer Rakoczy, Rosenheim (DE); Roderik Althoff, Rosenheim (DE)

Correspondence Address: SCOTT R. COX;LYNCH, COX, GILMAN & MAHAN, P.S.C., 500 WEST JEFFERSON STREET, SUITE 2100, LOUISVILLE, KY, 40202, US

(73) Assignee: SUD-CHEMIE AG, Munchen (DE)

(21) Appl. No.: 12/375,571

(22) PCT Filed: May 31, 2007
(86) PCT No.: PCT/EP2007/055321

§ 371 Date: Jan. 29, 2009

(30) Foreign Application Priority Data
Aug. 8, 2006 (DE) 10 2006 037 314.6

(51) Int. Cl. C07C 1/02; B01J 29/70

(52) U.S. Cl. 585/640; 502/73; 502/65

(57) ABSTRACT

The invention relates to the use of a catalyst based on crystalline aluminosilicates for the conversion of oxygenates to lower olefins, the catalyst having an SiO₂/Al₂O₃ molar ratio of 20 to 200 and being modified with 0.1% to 10% by weight of readily oxidant metal (calculated as the corresponding metal oxide) and/or with 0.05% to 5% by weight of cerium (calculated as Ce₂O₃), and also to an associated method.

The present invention relates to the use of a catalyst based on crystalline aluminosilicate which is modified by a metal with a slightly oxidizing effect and/or cerium when reacting oxygenates, such as methanol, ethanol, dimethylether or diethylether, to produce low olefins, such as propylene, and a corresponding process.

A typical conversion reaction for the use according to the invention and the process according to the invention is described by the following equation:

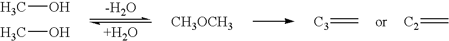

For the first step, the balanced reaction, a customary dehydrogenation catalyst, for example γ-aluminium oxide, or also a catalyst described within the framework of the present invention can be used as a so-called upstream catalyst. In principle, the reaction of the reaction mixture containing methanol and/or dimethylether vapour and water vapour can take place in a tubular reactor using an indirectly cooled catalyst, such as is described for example in EP 0 448 000 A1, which is to be included, by reference thereto, in the disclosure of the present invention. In particular the process described in line 28 on page 6 to line 8 on page 7 of EP 0 448 000 A1 is applied within the framework of the present invention, wherein the catalyst described within the framework of this invention is used.

In the second step, the conversion reaction to produce olefins, on the one hand the catalyst described within the framework of the present invention is used; on the other hand other zeolites-based catalysts can also be used. In principle, catalysts based on crystalline aluminosilicates of the pentasile type suitable for this are known from the state of the art.

Thus EP 1 424 128 A1 discloses for example such a catalyst which is built up from primary crystallites with an average diameter of at least 0.01 μm and less than 0.1 μm, which are at least 20% combined to produce agglomerates of 5 to 500 μm, wherein the primary crystallites or agglomerates are connected to one another by finely dispersed aluminium oxide whose BET surface is 300 to 600 m²/g and whose pore volume is 0.3 to 0.8 cm³/g, which is present in the H-form and in which the quantity of the finely dispersed aluminium oxide binder is 10 to 40 wt.-%, relative to the total weight of aluminosilicate and binder, wherein the finely dispersed aluminium oxide binder is present in the reaction mixture as peptizable hydrous aluminium oxide, wherein sodium aluminate is used as aluminium and alkali source and the primary synthesis of the crystalline aluminosilicate takes place without the addition of acid.

Furthermore, EP 0 369 364 A2 discloses such a catalyst with an Si/Al atomic ratio of at least 10, which is built up from primary crystallites with an average diameter of at least 0.1 μm and at most 0.9 μm, which are partly combined to produce agglomerates, wherein the primary crystallites or agglomerates are connected to one another by finely dispersed aluminium oxide which can be obtained by hydrolysis of organoaluminium compounds whose BET surface is 300 to 600 m²/g and whose pore volume is 0.3 to 0.8 cm³/g.

The disadvantage of these and other zeolite catalysts is that they tend towards reversible carbonization or irreversible dealuminization, which leads to a diffusion inhibition of the catalytic reaction or to a reduction in the intrinsic activity as far as the complete deactivation of the catalyst.

To examine the carbonization, the catalyst must be regenerated at regular intervals. This regeneration is carried out at temperatures between 500 and 800° C. and damages the zeolite catalyst in addition to the slow deactivation already proceeding under normal reaction conditions. Furthermore, the cycle times are shortened, and thus the effectiveness of the catalyst worsened, by the repeated regeneration.

In the case of irreversible dealuminization the catalyst is damaged, more precisely dealuminized, and thus deactivated, by the high water-vapour content of the process gas and by the high reaction temperature.

EP 0 955 080 A1 discloses a zeolite-based catalyst exclusively for the removal of nitrogen oxides from exhaust gases containing oxygen and water, which contains at least one metallic catalytic component, for example iron, and a process for its preparation.

The object of the present invention is to provide a catalyst suitable for the reaction of oxygenates to produce low olefins, which displays a slowed-down carbonization and a reduced hydrothermal dealuminization within the framework of this reaction.

Surprisingly, this object is achieved by the use of a catalyst modified by a metal with a slightly oxidizing effect and/or cerium, based on crystalline aluminosilicates, preferably of the pentasile type, in the reactions named at the outset.

The introduction of metals with a slightly oxidizing effect, for example iron, manganese, chromium or cobalt, in particular iron, has the surprising effect in the presence of water vapour, as present in the reactions named at the outset, that the formed coke is partly re-oxidized under the reaction conditions used without oxidizing the educts or products for their part. Thus the period of time until necessary regeneration is extended.

The introduction of cerium surprisingly leads to a clear increase in the remaining active centres in the case of a hydrothermal deactivation (dealuminization). For example, an increase by a factor of 2 to 3 is observed. The number of possible regeneration cycles thus increases by a comparable factor.

If both a metal with a slightly oxidizing effect, such as iron, manganese, chromium or cobalt, and also cerium are inserted into a zeolite catalyst, for example of the pentasile type, a synergistic effect results which leads, when reacting oxygenates to produce lower olefins, both to a lengthened cycle time and to an increased cycle count.

The introduction of one or more further metals from the group Zr, Ag, W, La and Th into zeolite catalysts leads to a further improvement in the reaction. Thus a combination of cerium and zircon for example shows a synergistic effect as regards the catalysis reaction, and a combination of iron and silver a synergistic effect as regards the hydrothermal stability of the catalyst.

In the state of the art zeolites which have an SiO₂/Al₂O₃ modulus (i.e. a molar ratio) of greater than 100 are customarily used for the described conversion reaction of oxygenates to produce low olefins. These zeolites are hydrothermally more stable compared with those with a lower SiO₂/Al₂O₃ modulus, but possess a lower initial activity. However, if zeolites with lower SiO₂/Al₂O₃ moduli are modified by a metal with a slightly oxidizing effect and/or cerium, these also show a good stability, whereby the number of possible regeneration cycles is increased. Consequently, zeolites with a SiO₂/Al₂O₃ modulus range below 100 are also suitable for the present invention. A molar ratio of 20 to 200, particularly preferably of 40 to 200, is preferred here.

The zeolites which can be used for the invention have for example an average pore diameter of 0.5 to 1 nm, preferably of 0.5 to 0.6 nm. Particularly preferred within the framework of the present invention are zeolites of the pentasile type which can have two different pore diameters, namely 0.54 and 0.57 nm. The pore diameters are determined crystallographically.

Zeolites which can be used for the invention include 0.1 to 10 wt.-%, preferably 0.5 to 2 wt.-% metal with a slightly oxidizing effect (calculated as corresponding metal oxide) and/or 0.05 to 5 wt.-%, preferably 0.1 to 1 wt.-% cerium (calculated as Ce₂O₃). Iron is preferred as a metal with a slightly oxidizing effect, wherein the wt.-% values then relate to Fe₂O₃. These and other weight percentages given within the framework of the present invention are based in each case, unless otherwise indicated in individual instances, on the total weight of all the solids.

The catalysts which can be used for the invention can additionally be modified by 0.1 to 1 wt.-%, preferably 0.1 to 0.5 wt.-% of a metal or several metals of the group consisting of Zr, Ag, W, La and Th.

The basis for the catalysts which can be used within the framework of the invention is described for example in EP 1 424 128 A1 and EP 0 369 364 A2. However, other zeolites customary in the trade, in particular of the pentasile type, can also be used for the preparation of these catalysts.

In any case the zeolite catalysts obtained or customary in the trade must also be modified by at least one metal with a slightly oxidizing effect and/or cerium in order to be suitable for the invention. This modification can in principle take place via exchange of solids ions, liquid ion exchange with aqueous metal salt solutions, or by impregnation.

The process described and claimed in EP 0 955 080 A1 is for example suitable for exchange of solids ions. In particular this comprises the following steps:
(A) introduction of iron and/or cerium and optionally Zr, Ag, W, La and/or Th into a synthetic zeolite material, wherein a dry mixture is prepared from the following components:
   component 1, consisting of ammonium salts, NH₃/NH₄ zeolites or N-containing compounds,
   component 2, consisting of highly siliceous zeolite structures with a SiO₂—Al₂O₃ ratio of 20 to 200,
   component 3, an active component selected from a compound of the group of active components named at the outset,
(B) mixing of components 1, 2 and 3 in a mill under normal pressure and at normal temperature and
(C) maintaining at a temperature of at least 300° C. until the ion exchange is complete,
(D) cooling to room temperature.

The introduction of iron into the zeolites by exchange of solids ions is also described in the periodicals Studies in Surface Science and Catalysis 69, pages 1641 to 1645 (1991) for zeolite Y and Studies in Surface Science and Catalysis 94, pages 665 to 672 for ZSM-5 zeolites. In the exchange of solids ions method for the preparation of the Fe zeolites by mechanical means mixtures of the NH₄- and/or H-form of the zeolites and an iron salt are produced by intensive mechanical mixing in a ball mill at room temperature. This mixture is then calcined in air in a chamber oven. After calcination the Fe-ZSM-5 zeolites are intensively washed in distilled water and dried after filtering-off of the zeolite.

In principle the following exchange of solids ions process can be used for the preparation of the catalysts within the framework of the present invention:
(a) provision of a crystalline aluminosilicate, preferably of the pentasile type;
(b1) introduction of metal with a slightly oxidizing effect and/or cerium into the aluminosilicate from step (a) by mixing the aluminosilicate with suitable compounds of the metal with a slightly oxidizing effect and/or suitable cerium compounds; or
(b2) introduction of metal with a slightly oxidizing effect into the aluminosilicate from step (a) followed by introduction of cerium into the product obtained in the first part-step or introduction of cerium into the aluminosilicate from step (a) followed by introduction of metal with a slightly oxidizing effect into the product obtained in the first part-step, in each case by mixing with suitable compounds of the metal with a slightly oxidizing effect or suitable cerium compounds; and
(b3) optionally, introduction of Zr, Ag, W, La and/or Th into the product obtained in step (b1) or (b2) by mixing with suitable compounds of Zr, Ag, W, La and/or Th;
(c) temperature treatment or calcination of the product obtained from step (b1), (b2) or (b3) (exchange of solids ions step);
(d) combination of the product from step (c) with 10 to 90 wt.-% (relative to the total quantity of ion-exchanged aluminosilicate) of a binder or of a mixture of single binders, selected from the group consisting of: aluminium- or silicon-based binders, aluminium oxide, hydrous aluminium oxide, SiO₂—, TiO₂—, WO₃— or ZrO₂ compounds; and
(e) temperature treatment of the product from step (d) at 400 to 700° C., preferably 450 to 600° C.

The preparation of crystalline aluminosilicate, preferably of the pentasile type, for step (a) is generally known. For example, it can be represented by the processes in EP 1 424 128 A1 or EP 0 369 364.

Steps (b1) and (b2) are preferably carried out by milling the aluminosilicate, preferably in its NH₄-form, with FeCl₂×4H₂O or CeCl₂×7H₂O, preferably under reductive conditions.

The necessary quantity of salt of metal with a slightly oxidizing effect or cerium used is at least sufficient for the end-product to be modified by 0.1 to 10 wt.-% metal with a slightly oxidizing effect (calculated as corresponding metal oxide) and/or by 0.05 to 5 wt.-% cerium (calculated as Ce₂O₃).

The preferred NH₄-form of the zeolite is obtained from the Na- or H-form by treatment with dilute ammonium salt solution, preferably NH₄NO₃ or (NH₄)₂SO₄ solution. For example, 10 to 100 g, preferably 20 to 80 g salt per litre water is used, wherein the zeolite is kept for 1 to 12 hours, preferably 2 to 4 hours, in this solution, for example accompanied by stirring, at a temperature of 20 to 100° C., preferably 50 to 80° C. The zeolite content of the solution is for example 10 to 30 wt.-%, preferably 15 to 25 wt.-%.

Step (b3) is preferably carried out by milling the product from step (b1) or (b2) with the halogen salts, preferably the chlorides and/or nitrates, of the named metals, preferably under reductive conditions.

Furthermore, step (c) is carried out preferably at 400 to 600° C. over 1 to 100 hours, preferably under reductive conditions, preferably with an oxygen content below the oxygen content of air (e.g. below 21 percent by volume or 23 percentage by mass). It is also conceivable to carry out step (c) under protective gas, such as NH₃ or H₂. For example, step (c) is carried out in a tray oven or batch oven within the framework of a discontinuous process, wherein a deep bed with a packing height of 5 to 20 cm is preferably used. Operation is for example at slightly below atmospheric pressure, for example 1 to 5% beneath atmospheric pressure.

To increase the concentration of the desired ions in the zeolite, i.e. of the metal with a slightly oxidizing effect and/or of the cerium and optionally of the metals selected from the group consisting of Zr, Ag, W, La and Th, steps (b1) or (b2) or (b3) and (c) can be repeated.

In order to bring the product from step (c) into a form suitable for the reaction of oxygenates to produce olefins, it is treated in step (d) with 10 to 90 wt.-%, preferably 15 to 30 wt.-% of an aluminium- or silicon-based binder (relative to the total mass of ion-exchanged aluminosilicate and binder). Preferred binders or mixtures of individual binders are selected from the group consisting of: aluminium- or silicon-based binders, aluminium oxide, hydrous aluminium oxide, SiO₂—, TiO₂—, WO₃— or ZrO₂ compounds. SiO₂ or its precursors are also suitable. The primary crystallites or the agglomerates are further preferably connected to one another by finely dispersed aluminium oxide or SiO₂, which can preferably be obtained by hydrolysis of organoaluminium or organosilicon compounds.

The product obtained according to step (d) is then calcined in step (e) at 400 to 700° C., preferably 450 to 600° C., in order to obtain a usable catalyst.

The liquid ion exchange to be used alternatively is carried out by introduction, preferably stirring-in of the aluminosilicate, preferably in its NH₄-form, into an aqueous solution of a salt of the metal with a slightly oxidizing effect, preferably into an iron salt solution, preferably into a FeSO₄ solution or FeCl₂, or into an aqueous cerium-salt solution, preferably into a Ce(SO₄)₂ solution. Suitable temperatures lie in the range from 20 to 100° C., preferably from 50 to 80° C. Suitable concentrations lie in the range from 1 to 10 wt.-%, preferably 2 to 8 wt.-% total solids content. Suitable residence times are in the range from 1 to 12 hours, preferably 2 to 4 hours. A suitable zeolite content in the solution is 10 to 30 wt.-%, preferably 15 to 25 wt.-%.

After the introduction of the metals by impregnation, aqueous ion exchange or by grinding with solid salts (exchange of solids ions) followed by temperature treatment the catalyst material can for example be used either in the form of pellets, as extrudate, as an extruded or coated honeycomb body.

The catalyst suitable for the invention is for example built up from primary crystallites with an average diameter of 0.01 to 0.9 μm. These primary crystallites are for example at least 20% combined to produce agglomerates of 5 to 500 μm, wherein the primary crystallites or agglomerates are connected to one another by an aluminium- or silicon-based binder. The primary crystallites preferably have an average diameter of 0.01 to 0.06 μm, in particular of 0.015 to 0.05 μm.

The average diameter of the primary crystallites is defined here as the arithmetic mean averaged over a variety of crystallites between the largest and the smallest diameter of an individual crystallite, determined using scanning electron microscopic examinations at a magnification of 80,000 (see below). This definition is significant in the case of crystallites with an irregular crystal habit, e.g. with rod-shaped crystallites. In the case of spherical or approximately-spherical crystallites the largest and the smallest diameter coincide.

The quoted values for the primary crystallites are the average dimensions (arithmetic mean of the largest and the smallest dimensions, obtained over a variety of crystallites). These values are determined with a LEO Field Emission Scanning Electron Microscope (LEO Electron Microscopy Inc, USA) using powder probes of the catalyst which had previously been redispersed in acetone, treated with ultrasound for 30 seconds and then deposited on a carrier (Probe Current Range: 4 pA to 10 nA). Measurement takes place at 80,000 magnification. The values could be confirmed at 253,000 magnification.

The BET surface of the catalyst which can be used for the present invention is for example 200 to 600 m²/g, preferably 250 to 400 m²/g (determined according to DIN 66 131) and its pore volume (determined by mercury porosimetry according to DIN 66 133; parameter specific total pore volume) 0.3 to 0.8 cm³/g.

The catalyst is also preferably in H-form.

The invention also relates to a process for the catalytic production of low olefins from oxygenates, wherein a catalyst is used which is based on crystalline aluminosilicate, preferably a zeolite of the pentasile type, and
has a SiO₂/Al₂O₃ molar ratio of 20 to 200 and
contains 0.1 to 10 wt.-% metal with a slightly oxidizing effect (calculated as corresponding metal oxide) and/or 0.05 to 5 wt.-% cerium (calculated as Ce₂O₃).

In this case, catalytic production of low olefins from oxygenates takes place through reaction of a mixture of oxygenate vapour and/or the vapour of the product obtained by splitting-off of at least one molecule of water from at least two molecules of oxygenate and water vapour and optionally additionally supplied water vapour in a tubular reactor using an indirectly cooled catalyst. In the case of methanol as oxygenate, by splitting-off one molecule of water from two molecules of methanol firstly dimethylether is thus produced, which is then converted into low olefins, for example ethylene (C2=) or propylene (C3=) using the catalyst described within the framework of the present invention.

Particular versions of this process arise from the corresponding dependent claims. The advantages associated therewith have already been described above within the framework of the use according to the invention.

The invention also further relates to the use of a catalyst based on crystalline aluminosilicate with a SiO₂/Al₂O₃ molar ratio from 20 to 200 which is modified by 0.1 to 10 wt.-% metal with a slightly oxidizing effect (calculated as corresponding metal oxide) and/or with 0.05 to 5 wt.-% cerium (calculated as Ce₂O₃), in organic synthesis reactions with oxygenates as educts and high water-vapour concentrations, wherein in the synthesis reactions there is a water-to-oxygenate molar ratio of 0.5 to 10. In these reactions there is preferably a water-to-oxygenate molar ratio of 2 to 4. In the above-described reaction of methanol to produce dimethylether and finally propylene or ethylene, there is for example a water-to-oxygenate molar ratio of 4. The catalysts suitable for such reactions correspond to those described above. A high water-to-oxygenate molar ratio is achieved for example by supplying additional water vapour to the reagents.

The advantages of the above-described catalysts within the framework of the present invention are shown using the following examples and the FIGURE.

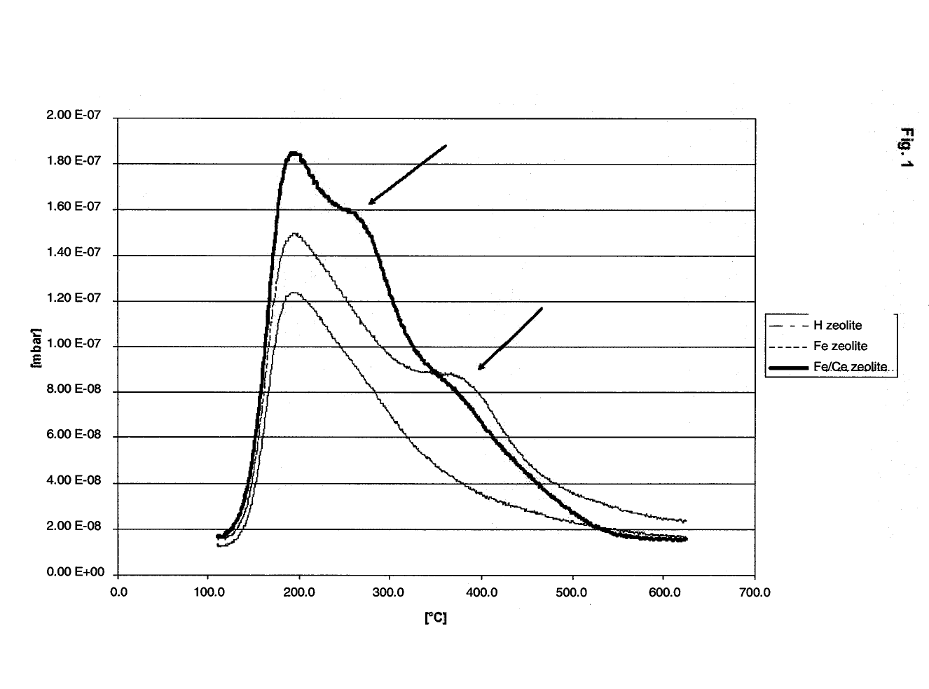

FIG. 1 shows the desorption curves of NH₃ at the H-, Fe- or Fe/Ce-modified zeolite, aged at 800° C., 10% water vapour in air for 24 hours in a tube furnace, of the following example 1.

### EXAMPLE 1

### Modification of a Zeolite by Iron or Iron and Cerium

Ammonium MFI type T 4480 from SüdChemie, Germany, was used as starting pentasile-type zeolite.

Modification by Iron:

1 kg of starting zeolite was milled at room temperature for one hour in a ball mill together with 25 g FeCl₂*4H₂O. The mixture was heated from room temperature to 550° C. in a chamber oven in air for 3 hours and kept there for 6 hours. After the mixture was cooled, a modified zeolite with 1 wt.-% Fe, calculated as Fe₂O₃, was obtained.

Modification by Iron and Cerium:

1 kg of starting zeolite was milled at room temperature for one hour together with 25 g FeCl₂*4H₂O and 11.4 g CeCl₃*7H₂O. The mixture was heated from room temperature to 550° C. in a chamber oven in air for 3 hours and kept there for 6 hours. After the mixture was cooled, a modified zeolite with 1 wt.-% Fe, calculated as Fe2O₃, and 0.5 wt.-% cerium, calculated as Ce₂O₃, was obtained.

### EXAMPLE 2

### Detection of the Increased Hydrothermal Stability of the Zeolite from Example 1 within the Framework of the Invention (Compared with a Zeolite not Modified by Metals)

It can be clearly seen from FIG. 1 that better results are achieved when using an Fe-modified zeolite, i.e. there is a better hydrothermal stability than when using the same zeolite in its H-form. In particular in the case of an Fe-modified zeolite the adsorption band (marked by an arrow) in the range from approx. 380 to 400° C. is present, but is missing in the H zeolite. This band lying at higher temperatures is however a measure of the hydrothermal stability of the zeolite, as the zeolite is clearly still able to adsorb NH₃ at these increased temperatures.

The results are even better when using a zeolite modified by both Fe and Ce. Here a clear adsorption band (marked by an arrow) can already be seen at approx. 280° C.

The partial pressure (mbar) of the mass 16, which was determined in an AMETEK mass spectrometer combined with a Zeton/Altamira adsorption/desorption apparatus AMI 200, is plotted on the Y axis of FIG. 1. The corresponding temperature (° C.) is given on the X axis.

To carry out the measurement, at 110° C. the probe is saturated with NH₃ after activation at 550° C. in the helium flow, and slowly heated to 750° C. after rinsing away the excess NH₃, and the NH₃ desorbing in the process is detected with the mass spectrometer (mass number 16).

1. A zeolite-based catalyst for the reaction of oxygenates to produce low olefins, characterized in that the catalyst
has a SiO₂/Al₂O₃ molar ratio of 20 to 200 and
is modified by 0.1 to 10 wt.-% metal with a slightly oxidizing effect (calculated as corresponding metal oxide) and/or with 0.05 to 5 wt.-% cerium (calculated as Ce₂O₃).

2. The catalyst according to claim 1, characterized in that the catalyst is additionally modified by 0.1 to 1 wt.-% of a metal of the group consisting of Zr, Ag, W, La and Th.

3. The catalyst according to claim 1, characterized in that the zeolite has an average pore diameter of 0.5 to 1 nm.

4. The catalyst according to claim 1, characterized in that the zeolite comprises a pentasile type zeolite.

5. The catalyst according to claim 1, characterized in that the catalyst can be obtained by the following steps:
(a) providing a crystalline aluminosilicate, preferably of the pentasile type;
(b1) introducing metal with a slightly oxidizing effect and/or cerium into the aluminosilicate from step (a) by mixing the aluminosilicate with suitable compounds of the metal with a slightly oxidizing effect and/or suitable cerium compounds; or
(b2) introducing metal with a slightly oxidizing effect into the aluminosilicate from step (a) followed by introduction of cerium into the product obtained in the first part-step or introduction of cerium into the aluminosilicate from step (a) followed by introduction of metal with a slightly oxidizing effect into the product obtained in the first part-step in each case by mixing with suitable compounds of the metal with a slightly oxidizing effect or suitable cerium compounds; and
(b3) optionally, introducing Zr, Ag, W, La and/or Th into the product obtained in step (b1) or (b2) by mixing with suitable compounds of Zr, Ag, W, La and/or Th;
(c) temperature treatment or calcination of the product obtained from step (b1), (b2) or (b3);
(d) combining the product from step (c) with 10 to 90 wt.-% (relative to the total quantity of ion-exchanged aluminosilicate) of a binder or a mixture of individual binders, selected from the group consisting of: aluminium- or silicon-based binders, aluminium oxide, hydrous aluminium oxide, SiO₂—, TiO₂—, WO₃— and ZrO₂ compounds; and
(e) temperature treatment of the product from step (d) at 400 to 700° C.

6. The catalyst according to claim 5, characterized in that iron (Fe) comprises the metal with a slightly oxidizing effect (calculation of the wt.-% values as Fe₂O₃).

7. The catalyst according to claim 1, characterized in that the oxygenate comprises methanol and/or dimethylether and the low olefin comprises propylene and/or ethylene.

8. Process for the catalytic production of low olefins from oxygenates, comprising providing a catalyst which is based on a crystalline aluminosilicate, that
has a SiO₂/Al₂O₃ molar ratio of 20 to 200, and
contains 0.1 to 10 wt.-% metal with a slightly oxidizing effect (calculated as corresponding metal oxide) and/or 0.05 to 5 wt.-% cerium (calculated as Ce₂O₃), and
passing the oxygenates over or through the catalyst.

9. Process according to claim 8, characterized in that the oxygenates comprise methanol and/or dimethylether and the low olefin comprise propylene and/or ethylene.

10. Process according to claim 8, characterized in that the catalyst is additionally modified by 0.1 to 1 wt.-% of a metal of the group consisting of Zr, Ag, W, La and Th.

11. Process according to claim 8, characterized in that the crystalline aluminosilicate has an average pore diameter of 0.5 to 1 nm.

12. Process according to claim 8, characterized in that the crystalline aluminosilicate comprises a pentasile type.

13. Process for the catalytic production of low olefins from oxygenates comprising:
(a) providing a crystalline aluminosilicate, preferably of the pentasile type; wherein the aluminosilicate
has a SiO₂/Al₂O₃ molar ratio of 20 to 200, and
contains 0.1 to 10 wt.-% metal with a slightly oxidizing effect (calculated as corresponding metal oxide) and/or 0.05 to 5 wt.-% cerium (calculated as Ce₂O₃),
(b1) wherein the metal with the slightly oxidizing effect and/or the cerium are introduced into the aluminosilicate from step (a) by mixing the aluminosilicate with suitable compounds of the metal with a slightly oxidizing effect and/or suitable cerium compounds; or
(b2) wherein the metal with the slightly oxidizing effect is introduced into the aluminosilicate from step (a) followed by introduction of cerium into the product obtained in the first part-step or introduction of cerium into the aluminosilicate from step (a) followed by introduction of metal with a slightly oxidizing effect into the product obtained in the first part-step in each case by mixing with suitable compounds of the metal with a slightly oxidizing effect or suitable cerium compounds; and
(b3) optionally, introducing Zr, Ag, W, La and/or Th into the product obtained in step (b1) or (b2) by mixing with suitable compounds of Zr, Ag, W, La and/or Th;
(c) temperature treatment or calcination of the product obtained from step (b1), (b2) or (b3) (exchange of solids ions step);
(d) combining the product from step (c) with 10 to 90 wt.-% (relative to the total quantity of ion-exchanged aluminosilicate) of a binder or a mixture of individual binders, selected from the group consisting of: aluminium- or silicon-based binders, aluminium oxide, hydrous aluminium oxide, SiO₂—, TiO₂—, WO₃— and ZrO₂ compounds; and
(e) temperature treatment of the product from step (d) at 400 to 700° C.

14. Process according to claim 13, characterized in that iron (Fe) is used as metal with a slightly oxidizing effect (calculation of the wt.-% values as Fe₂O₃).

15. Process of claim 13 further comprising an organic synthesis reaction with oxygenates as educts and high water-vapour concentrations, wherein there is a water-to-oxygenate molar ratio of 0.5 to 10 in the synthesis reaction.
